(19) United States
(12) Patent Application Publication
(10) Pub. No.: US 2024/0103008 A1
(43) Pub. Date: Mar. 28, 2024

(54) TREATMENT AND DETECTION METHODS FOR INFLAMMATORY BOWEL DISEASE

(71) Applicant: BRISTOL-MYERS SQUIBB COMPANY, Princeton, NJ (US)

(72) Inventors: Frederic Baribaud, Broomall, PA (US); Aisling Hall, Princeton, NJ (US); Jasmine Saini, Yardley, PA (US)

(73) Assignee: BRISTOL-MYERS SQUIBB COMPANY, Princeton, NJ (US)

(21) Appl. No.: 18/473,498

(22) Filed: Sep. 25, 2023

Related U.S. Application Data
(60) Provisional application No. 63/409,970, filed on Sep. 26, 2022.

(51) Int. Cl. G01N 33/68; A61K 45/06

(52) U.S. Cl. CPC G01N 33/6848; A61K 45/06; G01N 2800/065

(57) ABSTRACT

The present disclosure provides matched gut and peripheral biomarkers to assess, identify and improve therapeutic measures for IBD, in particular ulcerative colitis and Crohn's disease.

### CROSS REFERENCE TO RELATED APPLICATIONS

This application claims the benefit of U.S. Provisional Application Ser. No. 63/409,970 filed Sep. 26, 2022 which is incorporated herein in its entirety.

### FIELD

This invention relates to methods for the detection and treatment of inflammatory bowel disease, in particular ulcerative colitis and Crohn's disease.

### BACKGROUND OF THE INVENTION

Treatment of inflammatory bowel diseases (IBD) can be improved by the ability to identifying biomarkers, allowing the optimization of therapeutic dose and understand response. While molecular characterization from diseased tissues coupled with clinical measures of IBD response has identified patient pathotypes, enabling future tailoring of treatments to disease-driving mechanisms is still required. Most existing clinical datasets do not pair gut and peripheral samples and focus on well-defined IBD populations. Therefore, the generation of content-rich data sets from heterogeneous IBD populations is an outstanding effort worth pursuing.

### SUMMARY

The present disclosure provides matched gut and peripheral biomarkers to assess, identify and improve therapeutic measures for IBD, in particular ulcerative colitis and Crohn's disease. In some embodiements, disclosure provides a method for identifying and assessing the severity of Crohn's disease by measuring the gene expression of biomarkers in the blood and gut, in particular from colon and/or ileum, wherein the biomarker is selected from:

| Crohn's Disease | Ulcerative colitis |
|---|---|
| ADM | ADM |
| BMPR1B | BMPR1B |
| C10orf55 | C10orf55 |
| CATSPERB | CATSPERB |
| CD274 | CD274 |
| CLEC4D | CLEC4D |
| DSG3 | DSG3 |
| EIF5A2 | EIF5A2 |
| FCGR1A | FCGRIA |
| GAD1 | GAD1 |
| GALNT14 | GALNT14 |
| HLA-V | HLA-V |
| IDO1 | IDO1 |
| LYPD1 | LYPD1 |
| MFAP5 | SEMG1 |
| C2CD4A | C2CD4A |
| SEMG1 | CCNG1P1 |
| FCGR1BP | C2CD4B |
| OR2I1P | S100P |
| IGLV3-21 | OR2I1P |
| FCGR1CP | FCGR1B |
| C2CD4B | IGLV3-21 |
| IGLV3-27 | FCGR1C |
| G0S2 | PALM2 |
| S100P | GSEC |
| PALM2 | OSM |
| CCNG1P1 | PROK2 |
| GSEC | REG4 |
| EMG1 | S100A12 |
| OSM | S100A8 |
| PROK2 | S100A9 |
| REG4 | SLC16A14 |
| S100A12 | SLC26A4 |
| S100A8 | SLPI |
| S100A9 | SOCS3 |
| SLC16A14 | ST3GAL4 |
| SLC26A4 | TNFRSF6B |
| SLPI | UBD |
| SOCS3 | VNN1 |
| ST3GAL4 | |
| TNFRSF6B | |
| UBD | |
| VNN1 | |

In one embodiment, the disclosure provides a method for treating IBD in a patient suffering from or at risk of IBD, said method comprising: obtaining a sample of blood of the patient and measuring gene expression of biomarkers in blood; obtaining a tissue sample from said patient's gut and measuring gene expression of one ore more biomarkers selected from the lists above and administering a therapeutic agent for the treatment of IBD.

### DETAILED DESCRIPTION

### BRIEF DESCRIPTION OF FIGURES

FIG. 1: Illustrates IBD cohort sample and patient characteristics.

FIG. 2: Scatterplots of differentially expressed genes (DEGs) comparing UC to CD L vs NL tissue (left) and blood (right) are shown. The red and blue dots represent the significantly (p<0.05; Log2 FC≤1) up (red) or down (blue) regulated genes, respectively. The Venn diagram shows differentially up-regulated genes from the disease profile tissue comparisons. CD, Crohn's disease; UC, Ulcerative colitis; L, lesional; NL, non-lesional; IBD, inflammatory bowel disease; FC, fold change.

FIG. 3: Scatterplots of differentially expressed proteins (DEPs) comparing UC and CD L vs NL tissue (left) and L vs non-IBD blood (right). The Venn diagram shows differentially upregulated proteins from the various disease profile blood comparisons. CD, Crohn's disease; UC, Ulcerative colitis; L, lesional; NL, non-lesional; IBD, inflammatory bowel disease; FC, fold change.

FIG. 4: Weighted gene co-expression network analysis (WGCNA). WGCNA applied to the BMS IBD cohort (top) and to a published cohort (GSE16879) (bottom) are shown using the modules derived from the BMS IBD cohort and ssGSEA. The patient groups represented are based on the clinical severity (top) and the response status to an anti-TNF therapeutic divided into responders (R) and non-responders (NR) at baseline and post treatment (PostTr) (bottom). ssGSEA, single-sample gene set enrichment analysis.

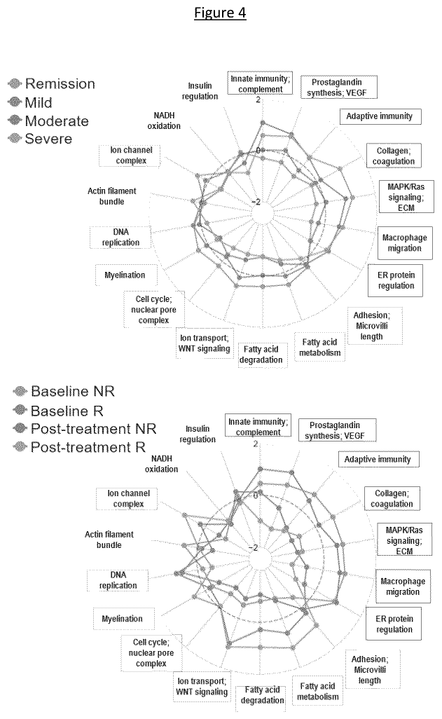

FIG. 5: A molecular IBD risk score captures clinically defined disease severity. Left, scatter plot showing the commonly and specific dysregulated genes from the BMS IBD cohort in tissue vs blood. Right, ssGSEA-based score using the commonly upregulated genes in tissue and blood as a signature (BMS T-B).

FIG. 6: Disease severity captured by the BMS IBD T-B signature followed by ssGSEA enrichment of the BMS IBD T-B signature (A) compared to other signatures capturing disease severity (B) and compared to cell-specific signatures (C) Immune related genes.

The present disclosure provides matched gut and peripheral biomarkers to assess, identify and improve therapeutic measures for IBD, in particular ulcerative colitis and Crohn's disease. In some embodiements, disclosure provides a method for identifying and assessing the severity of Crohn's disease by measuring the gene expression of biomarkers in the blood and gut, in particular from colon and/or ileum, wherein the biomarker is selected from:

| Crohn's Disease | Ulcerative colitis |
|---|---|
| ADM | ADM |
| BMPR1B | BMPR1B |
| C10orf55 | C10orf55 |
| CATSPERB | CATSPERB |
| CD274 | CD274 |
| CLEC4D | CLEC4D |
| DSG3 | DSG3 |
| EIF5A2 | EIF5A2 |
| FCGR1A | FCGR1A |
| GAD1 | GAD1 |
| GALNT14 | GALNT14 |
| HLA-V | HLA-V |
| IDO1 | IDO1 |
| LYPD1 | LYPD1 |
| MFAP5 | SEMG1 |
| C2CD4A | C2CD4A |
| SEMG1 | CCNG1P1 |
| FCGR1BP | C2CD4B |
| OR2I1P | S100P |
| IGLV3-21 | OR2I1P |
| FCGR1CP | FCGRIB |
| C2CD4B | IGLV3-21 |
| IGLV3-27 | FCGR1C |
| G0S2 | PALM2 |
| S100P | GSEC |
| PALM2 | OSM |
| CCNG1P1 | PROK2 |
| GSEC | REG4 |
| EMG1 | S100A12 |
| OSM | S100A8 |
| PROK2 | S100A9 |
| REG4 | SLC16A14 |
| S100A12 | SLC26A4 |
| S100A8 | SLPI |
| S100A9 | SOCS3 |
| SLC16A14 | ST3GAL4 |
| SLC26A4 | TNFRSF6B |
| SLPI | UBD |
| SOCS3 | VNN1 |
| ST3GAL4 | |
| TNFRSF6B | |
| UBD | |
| VNN1 | |

In one embodiment, the disclosure provides a method for treating IBD in a patient suffering from or at risk of IBD, said method comprising: obtaining a sample of blood of the patient and measuring gene expression of biomarkers in blood; obtaining a tissue sample from said patient's gut and measuring gene expression of one ore more biomarkers selected from the lists above and administering a therapeutic agent for the treatment of IBD. In some embodiments, IBD is selected from Crohn's disease and ulcerative colitis. In some embodiments, the expression of biomarker is measured in the sample is at least about 10% of expression of said biomarker measured in a control sample from a subject not suffering from Crohn's disease. In some ebmodiments, the therapeutic agent is selected from the group consisting of: a vitamin supplement, an anti-inflammatory, a corticosteroid, a 5-aminosalicylate, an immunosuppressant, azathioprine, mercaptopurine, an anti-TNF-α antibody, infliximab, adalimumab, certolizumab pegol, methotrexate, an anti-α 4-integrin antibody, natalizumab, vedolizumab, an anti-interleukin antibody, ustekinumab, an antibacterial antibiotic, ciprofloxacin, and metronidazole. In some embodiments, the therapeutic agent is selected from the group consisting of: vitamin B12, vitamin D, calcium, certolizumab pegol, methotrexate, and natalizumab.

In one embodiment, provided is a method of predicting a response of a subject diagnosed with an IBD to an anti-interleukin (IL) treatment of the IBD, the method comprising: contacting a sample from a subject with a set of probes capable of detecting a panel of biomarkers selected from the group the list above; and determining a pattern of the panel of biomarkers; wherein the pattern of the panel of biomarkers predicts the response to the anti-IL treatment in the subject.

In some embodiments, the anti-inflammatory treatment provided herein is an anti-tumor necrosis factor (TNF) treatment, a JAK inhibitor (JAKi) treatment, or an anti-interleukin (IL) treatment. In some embodiments, the anti-inflammatory treatment is an anti-IL-23 or anti-IL-12/23 treatment. In other embodiments, the anti-IL treatment is ustekinumab. In some embodiments, the anti-inflammatory treatment is the JAK inhibitor treatment. In other embodiments, the anti-inflammatory treatment is the anti-TNF treatment. In some embodiments, the anti-TNF treatment is golimumab.

In some embodiments, the anti-inflammatory treatment provided herein for the method of treating the subject diagnosed with IBD, is an anti-tumor necrosis factor (TNF) treatment, a JAK inhibitor (JAKi) treatment, or an anti-interleukin (IL) treatment. In some embodiments, the anti-inflammatory treatment is an anti-IL-23 or anti-IL-12/23 treatment. In other embodiments, the anti-IL treatment is ustekinumab. In some embodiments, the anti-inflammatory treatment is the JAK inhibitor treatment. In other embodiments, the anti-inflammatory treatment is the anti-TNF treatment. In some embodiments, the anti-TNF treatment is golimumab.

In another aspect, the kit provided herein comprises a set of isolated probes capable of detecting one or more biomarkers selected from Table A:

TABLE A
ADM
BMPR1B
C10orf55
CATSPERB
CD274
CLEC4D
DSG3
EIF5A2
FCGRIA
GAD1
GALNT14
HLA-V
IDO1
LYPD1
MFAP5
C2CD4A
SEMG1
FCGR1BP
OR2I1P
IGLV3-21
FCGR1CP
C2CD4B
IGLV3-27
G0S2
S100P
PALM2
CCNG1P1
GSEC
EMG1
OSM
PROK2
REG4
S100A12
S100A8
S100A9
SLC16A14
SLC26A4
SLPI
SOCS3
ST3GAL4
TNFRSF6B
UBD
VNN1
CCNG1P1
FCGR1C

In another aspect, the kit provided herein comprises a set of isolated probes capable of detecting at least five, preferably ten, or more preferably fifteen or twenty or twenty five or thirty, or more biomarkers selected from Table A.

Unless defined otherwise, all technical and scientific terms used herein have the same meaning as commonly understood to one of ordinary skill in the art to which this invention pertains. Otherwise, certain terms used herein have the meanings as set forth in the specification.

It must be noted that as used herein and in the appended claims, the singular forms “a,” “an,” and “the” include plural reference unless the context clearly dictates otherwise.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the invention.

As used herein, the terms “comprises,” “comprising,” “includes,” “including or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

It should also be understood that the terms “about,” “approximately,” “generally,” “substantially” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

The term “expressed” or “expression” as used herein refers to the transcription from a gene to give an RNA nucleic acid molecule at least complementary in part to a region of one of the two nucleic acid strands of the gene. The term “expressed” or “expression” as used herein also refers to the translation from the RNA molecule to give a protein, a polypeptide, or a portion thereof.

As used herein, “biomarker” refers to a gene or protein whose level of expression or concentration in a sample is altered compared to that of a normal or healthy sample or is indicative of a condition. The biomarkers disclosed herein are genes and/or proteins whose expression level or concentration or timing of expression or concentration correlates with the prognosis of an inflammatory bowel disease, in particular, ulcerative colitis and/or Crohn's disease.

The term “administering” with respect to the methods of the invention, means a method for therapeutically or prophylactically preventing, treating or ameliorating a syndrome, disorder or disease (e.g., an inflammatory bowel disease (IBD)) as described herein. Such methods include administering an effective amount of said therapeutic agent at different times during the course of a therapy or concurrently in a combination form. The methods of the invention are to be understood as embracing all known therapeutic treatment regimens.

The term “effective amount” or “therapeutically effective amount” means that amount of active compound or pharmaceutical agent, a combination of therapeutic compounds or pharmaceutical compositions thereof provided herein, that elicits the biological or medicinal response in a tissue system, animal or human, that is being sought by a researcher, veterinarian, medical doctor, or other clinician, which includes preventing, treating or ameliorating a syndrome, disorder, or disease being treated, or the symptoms of a syndrome, disorder or disease being treated (e.g., IBD).

### EXAMPLES

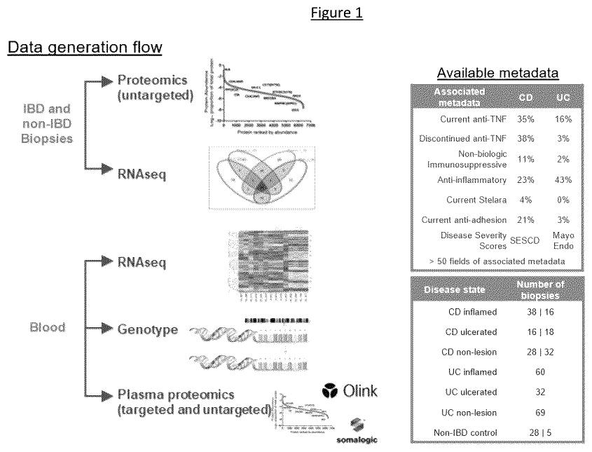

Endoscopic biopsies were obtained from 39 Crohn's disease (CD) patients, 59 Ulcerative colitis (UC) patients, and 15 non-IBD donors from multiple matched colon and ileum sites (FIG. 1). The tissue biopsies were immediately treated with RNAlater and stored at −80 freezer. RNA, DNA and protein were co-extracted from the frozen biopsies using Qiagen All-Prep DNA/RNA/Protein Kit. RNA was sequenced using Total RNAseq method with rRNA and globin depletion. Protein extract was profiled by mass spectrometry-based proteomics (LC-MS). Peripheral whole blood was used for generating DNA, RNA, and plasma followed by RNAseq, LC-MS proteomics along with untargeted SOMAscan plasma proteomics. DNA samples from both tissue and blood were used for genotyping with Illumina array chip Omni2.5-8 kit v1.3.

Raw reads were aligned to the human genome with STAR aligner. Gene level counts data was generated using RSEM. Statistical analysis of differential gene expression was performed on transcriptome and proteome from gut and peripheral samples, using linear regression model (LIMMA package in R). Between sample normalization was performed using EgdeR TMM, followed by linear modeling of gene expression using limma/voom workflow to identify differentially expressed genes. Coexpression analysis was performed using Weighted Gene Coexpression Network Approach (WGCNA) on top 11,000 differentially expressed genes to identify disease phenotype.

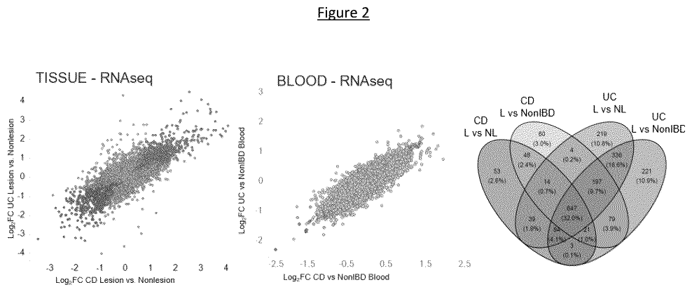

Transcriptomic disease profiles were identified from tissue and blood (FIG. 2). No differences in CD vs. UC were observed from either biopsy or blood (data not shown). The biopsies revealed significantly upregulated metabolic and immune pathways in both lesional (L) vs. non-lesional (NL), and in IBD vs. non-IBD controls. Further pathway enrichment analysis identified upregulation of genes involved in inflammatory response, cytokine production and regulation of collagen in tissue. Downregulated genes were involved in glycoprotein and triglyceride metabolic processes. In blood, immune related pathways were upregulated while regulation of actin cytoskeleton and negative regulation of DCs were downregulated.

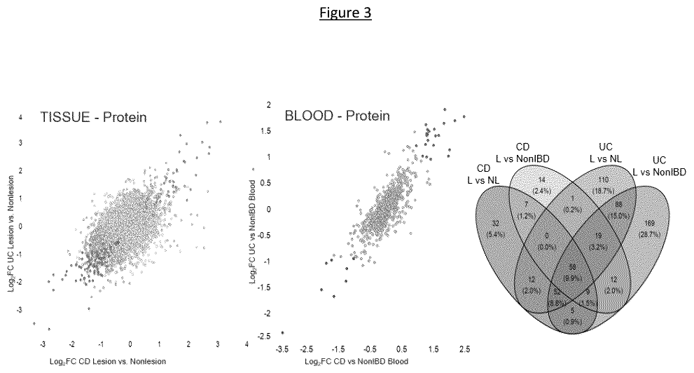

Unbiased LCMS identified expression of 7000 proteins in the tissue biopsies (FIG. 3). Some of the pathways identified in the upregulated DEPs were neutrophil degranulation, chemokine production, negative regulation of epithelial cell differentiation and chaperone proteins involved in post translational protein modifications. The downregulated DEPs included e.g., proteins involved with mitochondrial activity, cadherin junctions, fatty acid degradation and ketone metabolism.

800 proteins were detected in blood using an unbiased LCMS proteomics approach. The upregulated DEPs were involved in Neutrophil/Granulocyte migration while the downregulated DEPS were involved in collagen binding. These results were confirmed using in independent proteomics platform (SomaScan®, SomaLogic).

The DEGs from the BMS IBD cohort clustered into 18 modules. Modules divided into three groups: 1) the more severe the disease the more enriched (7 modules—green box), 2) the less severe the more enriched (8 modules—orange box) and 3) no enrichment difference across severities.

Looking at the BMS IBD module enrichments in the GSE16879 data sets (bottom) we observe that: 1) many modules change in R only when comparing baseline to PostTr, 2) most of the modules show a differential enrichment at baseline between R and NR and 3) some modules show differences at baseline between R and NR such as innate immune

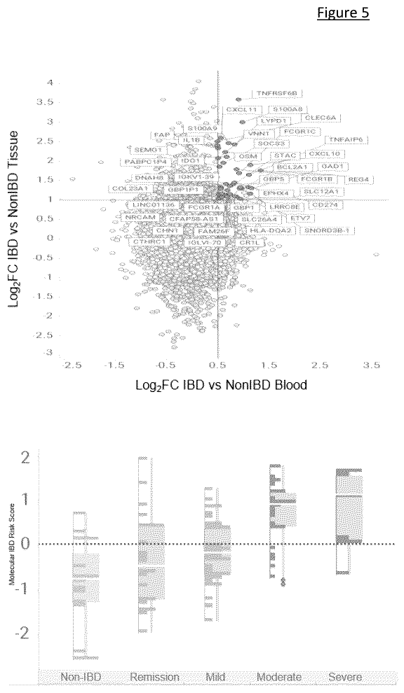

We identified shared markers in tissue and blood allowing for development of a common signature (BMS_IBD_T-B) which tracks with clinical severity of colon biopsies (FIG. 5).

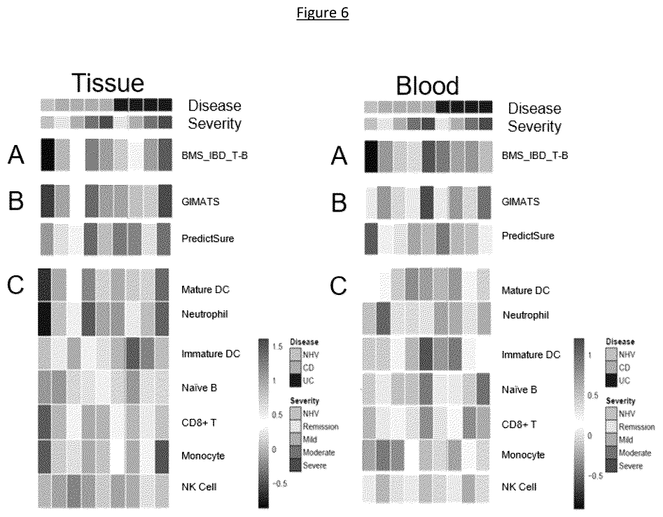

The BMS_IBD_T-B signature in blood better distinguishes clinical disease severity compared to GIMATS (Reference: Martin J, Chang C, Boschetti G et al. Single-Cell Analysis of Crohn's Disease Lesions Identifies a Pathogenic Cellular Module Associated with Resistance to Anti-TNF Therapy. Cell. 2019 Sep. 5; 178(6): 1493-1508) or the PredictSure blood test (Biasci D, Lee J, Noor N et al. A blood-based prognostic biomarker in IBD. Gut 2019;68:1386-1395) while having a comparable performance in tissue (FIG. 6A vs 6B).

The BMS_IBD_T-B signature in tissue has similar enrichment scores as the mature DC and neutrophil signatures in tissue (FIG. 6A vs 6C). These 3 signatures correlate to clinical disease severity unlike the other cell specific signatures. The CD8T, monocyte and NK cell signatures show highest enrichment only in severe tissue samples.

In blood the best disease severity correlated signatures other than the BMS_IBD_T-B signature are also the Mature DC and Neutrophil signatures while no other cell signature show a disease severity correlated enrichment.

We claim:

1. A biomarker for diagnosing ulcerative colitis or Crohn's disease in a patient suffering from or at risk of inflammatory bowel disease, through measuring gene expression of biomarkers in blood and in gut tissue of said patient, wherein said biomarker is selected from:

| Crohn's Disease | Ulcerative colitis |
|---|---|
| ADM | ADM |
| BMPR1B | BMPR1B |
| C10orf55 | C10orf55 |
| CATSPERB | CATSPERB |
| CD274 | CD274 |
| CLEC4D | CLEC4D |
| DSG3 | DSG3 |
| EIF5A2 | EIF5A2 |
| FCGR1A | FCGR1A |
| GAD1 | GAD1 |
| GALNT14 | GALNT14 |
| HLA-V | HLA-V |
| IDO1 | IDO1 |
| LYPD1 | LYPD1 |
| MFAP5 | SEMG1 |
| C2CD4A | C2CD4A |
| SEMG1 | CCNG1P1 |
| FCGR1BP | C2CD4B |
| OR2I1P | S100P |
| IGLV3-21 | OR211P |
| FCGR1CP | FCGR1B |
| C2CD4B | IGLV3-21 |
| IGLV3-27 | FCGR1C |
| G0S2 | PALM2 |
| S100P | GSEC |
| PALM2 | OSM |
| CCNG1P1 | PROK2 |
| GSEC | REG4 |
| EMG1 | S100A12 |
| OSM | S100A8 |
| PROK2 | S100A9 |
| REG4 | SLC16A14 |
| S100A12 | SLC26A4 |
| S100A8 | SLPI |
| S100A9 | SOCS3 |
| SLC16A14 | ST3GAL4 |
| SLC26A4 | TNFRSF6B |
| SLPI | UBD |
| SOCS3 | VNN1 |
| ST3GAL4 | |
| TNFRSF6B | |
| UBD | |
| VNN1 | |

2. A method of treating Crohn's disease in a patient suffering from or at risk of inflammatory bowel disease, said method comprising: obtaining a sample of blood of the patient and measuring gene expression of biomarkers in blood; obtaining a tissue sample from said patient's gut and measuring gene expression of biomarkers; wherein said biomarker is selected from:
ADM
BMPR1B
C10orf55
CATSPERB
CD274
CLEC4D
DSG3
EIF5A2
FCGR1A
GAD1
GALNT14
HLA-V
IDO1
LYPD1
MFAP5
C2CD4A
SEMG1
FCGR1BP
OR2I1P
IGLV3-21
FCGR1CP
C2CD4B
IGLV3-27
G0S2
S100P
PALM2
C CNG1P1
GSEC
EMG1
OSM
PROK2
REG4
S100A12
S100A8
S100A9
SLC16A14
SLC26A4
SLPI
SOCS3
ST3GAL4
TNFRSF6B
UBD
VNN1
and administering one or more therapeutic agents for the treatment of Crohn's disease;
wherein the expression of said biomarkers is elevated in said patient.

3. The method of claim 2, wherein the expression of biomarker is measured in the sample is no more than about 10% of expression of said biomarker measured in a control sample from a subject not suffering from Crohn's disease.

4. The method of claim 2, wherein said therapeutic agent is selected from the group consisting of: a vitamin supplement, an anti-inflammatory, a corticosteroid, a 5-aminosalicylate, an immunosuppressant, azathioprine, mercaptopurine, an anti-TNF-α antibody, infliximab, adalimumab, certolizumab pegol, methotrexate, an anti-α 4-integrin antibody, natalizumab, vedolizumab, an anti-interleukin antibody, ustekinumab, an antibacterial antibiotic, ciprofloxacin, and metronidazole.

5. The method of claim 2, wherein said therapeutic agent is selected from the group consisting of: vitamin B12, vitamin D, calcium, certolizumab pegol, methotrexate, and natalizumab.

6. A method of diagnosing and treating ulcerative colitis in a patient suffering from or at risk of inflammatory bowel disease, said method comprising: obtaining a sample of blood of the patient; measuring expression of biomarkers in blood wherein said biomarker is selected from:
ADM
BMPR1B
C10orf55
CATSPERB
CD274
CLEC4D
DSG3
EIF5A2
FCGR1A
GAD1
GALNT14
HLA-V
IDO1
LYPD1
SEMG1
C2CD4A
CCNG1P1
C2CD4B
S100P
OR2I1P
FCGR1B
IGLV3-21
FCGR1C
PALM2
GSEC
OSM
PROK2
REG4
S100A12
S100A8
S100A9
SLC16A14
SLC26A4
SLPI
SOCS3
ST3GAL4
TNFRSF6B
UBD
VNN1
and administering one or more therapeutic agents for the treatment of ulcerative colitis;
wherein the expression of biomarkers selected from claim 2 is elevated in said patient.

7. The method of claim 2, wherein the expression of biomarker is measured in the sample is no more than about 10% of expression of said biomarker measured in a control sample from a subject not suffering from ulcerative colitis disease.

8. The method of claim 2, wherein said therapeutic agent is selected from the group consisting of: a vitamin supplement, an anti-inflammatory, a corticosteroid, a 5-aminosalicylate, an immunosuppressant, azathioprine, mercaptopurine, an anti-TNF-α antibody, infliximab, adalimumab, certolizumab pegol, methotrexate, an anti-α 4-integrin antibody, natalizumab, vedolizumab, an anti-interleukin antibody, ustekinumab, an antibacterial antibiotic, ciprofloxacin, and metronidazole.

9. The method of claim 2, wherein said therapeutic agent is selected from the group consisting of: vitamin B12, vitamin D, calcium, certolizumab pegol, methotrexate, and natalizumab.

10. A method of treating IBD comprising the step of administering one or more therapeutic agents to a patient in need thereof; wherien said therapeutic agent is selected form vitamin supplement, an anti-inflammatory, a corticosteroid, a 5-aminosalicylate, an immunosuppressant, azathioprine, mercaptopurine, an anti-TNF-α antibody, infliximab, adalimumab, certolizumab pegol, methotrexate, an anti-α 4-integrin antibody, natalizumab, vedolizumab, an anti-interleukin antibody, ustekinumab, an antibacterial antibiotic, ciprofloxacin, and metronidazole; and wherien said patient is diagnosed to have IBD by determing gene expression of biomarkers in blood and in gut tissue, wherein said biomarker is selected from:

| Crohn's Disease | Ulcerative colitis |
|---|---|
| ADM | ADM |
| BMPR1B | BMPR1B |
| C10orf55 | C10orf55 |
| CATSPERB | CATSPERB |
| CD274 | CD274 |
| CLEC4D | CLEC4D |
| DSG3 | DSG3 |
| EIF5A2 | EIF5A2 |
| FCGR1A | FCGR1A |
| GAD1 | GAD1 |
| GALNT14 | GALNT14 |
| HLA-V | HLA-V |
| IDO1 | IDO1 |
| LYPD1 | LYPD1 |
| MFAP5 | SEMG1 |
| C2CD4A | C2CD4A |
| SEMG1 | CCNG1P1 |
| FCGR1BP | C2CD4B |
| OR2I1P | S100P |
| IGLV3-21 | OR2I1P |
| FCGR1CP | FCGR1B |
| C2CD4B | IGLV3-21 |
| IGLV3-27 | FCGR1C |
| G0S2 | PALM2 |
| S100P | GSEC |
| PALM2 | OSM |
| CCNG1P1 | PROK2 |
| GSEC | REG4 |
| EMG1 | S100A12 |
| OSM | S100A8 |
| PROK2 | S100A9 |
| REG4 | SLC16A14 |
| S100A12 | SLC26A4 |
| S100A8 | SLPI |
| S100A9 | SOCS3 |
| SLC16A14 | ST3GAL4 |
| SLC26A4 | TNFRSF6B |
| SLPI | UBD |
| SOCS3 | VNN1 |
| ST3GAL4 | |
| TNFRSF6B | |
| UBD | |
| VNN1 | |
